(19) United States
(12) Patent Application Publication
(10) Pub. No.: US 2019/0117041 A1
(43) Pub. Date: Apr. 25, 2019

(54) MEDICAL EQUIPMENT AND MEDICAL EQUIPMENT SYSTEM

(71) Applicant: OLYMPUS CORPORATION, Tokyo (JP)

(72) Inventors: Satoshi TANAKA, Tokyo (JP); Yoichiro SAKANOUE, Tokyo (JP)

(73) Assignee: OLYMPUS CORPORATION, Tokyo (JP)

(21) Appl. No.: 16/230,034

(22) Filed: Dec. 21, 2018

Related U.S. Application Data
(63) Continuation of application No. PCT/JP2017/017834, filed on May 11, 2017.

(30) Foreign Application Priority Data
Sep. 28, 2016 (JP) 2016-189943

(51) Int. Cl. A61B 1/00; H04N 5/225; A61B 1/06

(52) U.S. Cl. CPC A61B 1/00006; H04N 5/2256; A61B 1/00009; A61B 1/05; A61B 1/0669; H04N 2005/2255; A61B 1/0002

(57) ABSTRACT

A processor includes: a table configured to retain first control parameter information for controlling operations of a light source section; a reading circuit configured to read information stored in a memory from an endoscope including the memory that is capable of storing second control parameter information for controlling the operations of the light source section or the like; and a determination circuit configured to determine whether or not the reading circuit can read the second control parameter information. When it is determined that the second control parameter information can be read, the second control parameter information read by the reading circuit is outputted. When it is determined that the second control parameter information cannot be read, the first control parameter information retained by the table is outputted.

### CROSS REFERENCE TO RELATED APPLICATION

This application is a continuation application of PCT/JP2017/017834 filed on May 11, 2017 and claims benefit of Japanese Application No. 2016-189943 filed in Japan on Sep. 28, 2016, the entire contents of which are incorporated herein by this reference.

### BACKGROUND OF THE INVENTION

### 1. Field of the Invention

The present invention relates to medical equipment and a medical equipment system and, more particularly, to medical equipment used in combination with an endoscope and a medical equipment system.

### 2. Description of the Related Art

Conventionally, a medical equipment system in which a plurality of pieces of medical equipment are combined is widely used. For example, an endoscope system configured by connecting an endoscope and a main body unit such as a video processor is among such systems.

The endoscope system includes a light source section configured to output illuminating light that illuminates an object. The light source section is controlled so as to output the illuminating light of a light quantity or the like suitable for the endoscope connected to the main body unit.

For example, in the description of Japanese Patent No. 5922209, an endoscope system is proposed, which includes a plurality of light sources and in which a processor reads endoscope identification information (that is, an endoscope ID) stored in an endoscope, refers to a table in which the endoscope ID is associated with light quantity ratio information on the plurality of light sources, and controls the plurality of light sources suitably for the endoscope.

Japanese Patent Application Laid-Open Publication No. 2009-39432 discloses an endoscope apparatus in which an endoscope includes a storage section storing illumination time information on illuminating light, and a processor uses the illumination time information to control a light source section.

### SUMMARY OF THE INVENTION

Medical equipment according to an aspect of the present invention is medical equipment used in combination with an endoscope and includes: a table configured to retain first control parameter information for controlling operations of a light source section or an image processing section; a reading circuit configured to read information stored in a memory from the endoscope including the memory that is capable of storing second control parameter information for controlling the operations of the light source section or the image processing section; a determination circuit configured to determine whether or not the second control parameter information can be read from the memory; and an output circuit configured to output the second control parameter information read by the reading circuit when the determination circuit determines that the second control parameter information can be read from the memory, and to output the first control parameter information retained by the table when the determination circuit determines that the second control parameter information cannot be read from the memory.

A medical equipment system according to another aspect of the present invention includes: an endoscope; and medical equipment used in combination with the endoscope, wherein the medical equipment includes: a table configured to retain first control parameter information for controlling operations of a light source section or an image processing section; a reading circuit configured to read information stored in a memory from the endoscope including the memory that is capable of storing second control parameter information for controlling the operations of the light source section or the image processing section; a determination circuit configured to determine whether or not the second control parameter information can be read from the memory; and an output circuit configured to output the second control parameter information read by the reading circuit when the determination circuit determines that the second control parameter information can be read from the memory, and to output the first control parameter information retained by the table when the determination circuit determines that the second control parameter information cannot be read from the memory.

Medical equipment according to still another aspect of the present invention is medical equipment used in combination with an endoscope and includes: a table configured to retain first control parameter information for controlling operations of a light source section or an image processing section; and a processor including hardware, wherein the processor is configured to: read information stored in a memory from the endoscope including the memory that is capable of storing second control parameter information for controlling the operations of the light source section or the image processing section; determine whether or not the second control parameter information can be read from the memory; and output the read second control parameter information when it is determined that the second control parameter information can be read from the memory, and output the first control parameter information retained by the table when it is determined that the second control parameter information cannot be read from the memory.

### BRIEF DESCRIPTION OF THE DRAWINGS

FIG. 1 is a block diagram showing a configuration of an endoscope system according to an embodiment of the present invention;

FIG. 2 is a diagram showing an example of information stored in a memory 13, according to the embodiment of the present invention;

FIG. 3 is a waveform diagram of a drive signal supplied to each light emitting portion in an ordinary light observation mode in case of a simultaneous method, according to the embodiment of the present invention;

FIG. 4 is a waveform diagram of a drive signal supplied to each light emitting device in a narrow-band light observation mode in case of the simultaneous method, according to the embodiment of the present invention; and

FIG. 5 is a flowchart showing an example of a flow of operations of a processor, according to the embodiment of the present invention.

### DETAILED DESCRIPTION OF THE PREFERRED EMBODIMENT(S)

Hereinafter, an embodiment of the present invention will be described with reference to drawings.

### (Configuration)

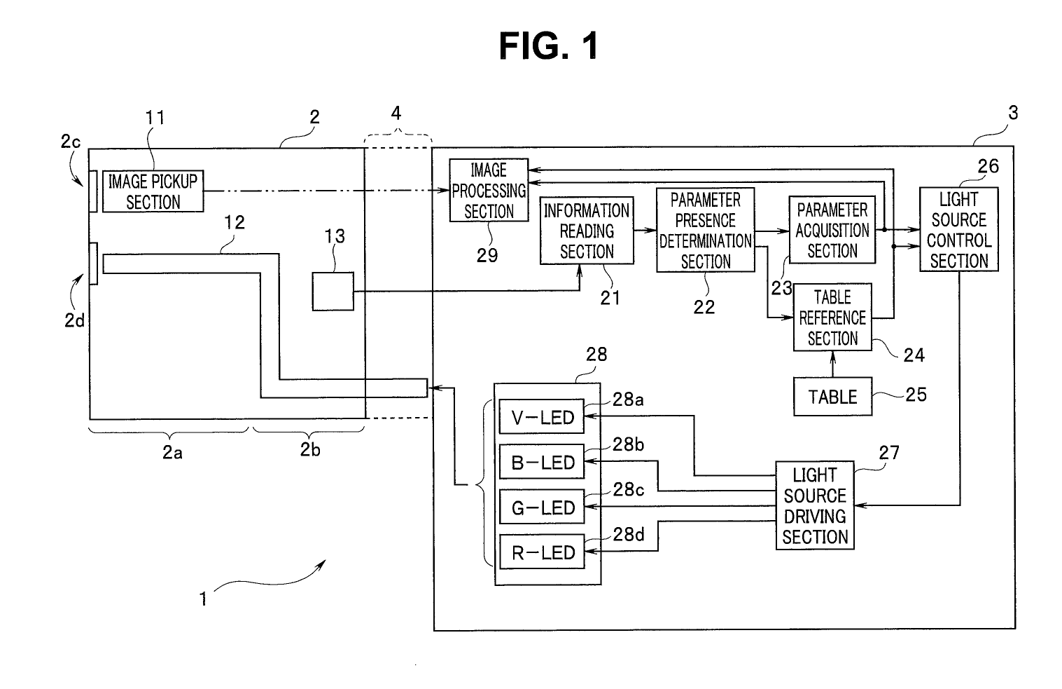

FIG. 1 is a block diagram showing a configuration of an endoscope system according to the present embodiment. Note that FIG. 1 shows only partial constituent elements of the endoscope system, and constituent elements other than the constituent elements described below are not shown and omitted.

The endoscope system 1 is a medical equipment system including an endoscope 2 and a processor 3 as a main body unit. The endoscope 2 includes an elongated insertion portion 2a and an operation portion 2b to which a proximal end portion of the insertion portion 2a is connected. The endoscope 2 is connected to the processor 3 through a cable 4 (indicated by a dotted line) extending from the operation portion 2b of the endoscope 2. The processor 3 and the cable 4 are connected by a connector. That is, the processor 3 is medical equipment used in combination with the endoscope 2.

The endoscope system 1 has two observation modes, namely, an ordinary light observation mode and a narrow-band light observation mode. A user can observe the inside of a body of a subject in a desired one of the observation modes by operating an operation button (not shown).

An observation window 2c and an illumination window 2d are provided at a distal end portion of the insertion portion 2a of the endoscope 2.

The endoscope 2 includes an image pickup section 11, a light guide 12, and a memory 13. The image pickup section 11 is disposed behind the observation window 2c. The image pickup section 11 includes an image pickup device, which receives light from the observation window 2c provided at the distal end portion of the insertion portion 2a and performs photoelectric conversion. The image pickup device is, for example, a CMOS sensor. An image signal of an image acquired through image pickup by the image pickup section 11 is outputted to an image processing section 29 of the processor 3 through a signal line, as indicated by a dash-double-dot line.

The light guide 12 is inserted into the insertion portion 2a, the operation portion 2b, and the cable 4, and a distal end of the light guide 12 is disposed behind the illumination window 2d and fixed. A proximal end of the light guide 12 is disposed and fixed to a proximal end portion of the cable 4 so that light from a light source section 28, which will be described later, enters the proximal end of the light guide 12.

The memory 13 is a non-volatile memory such as a ROM or a flash memory. The memory 13 can store at least one of endoscope identification information (hereinafter referred to as the endoscope ID) and control parameter information. The control parameter information is information for controlling operations of each section or portion when the endoscope system 1 operates.

Note that the memory 13 may be provided within the connector of the cable 4 for connecting to the processor 3.

Note further that the endoscope 2 includes other constituent elements such as an operation button, a bending mechanism, and a bending operation portion, which are not shown in FIG. 1, and a description of the elements is omitted.

The processor 3 to which the endoscope 2 is connected includes an information reading section 21, a parameter presence determination section 22, a parameter acquisition section 23, a table reference section 24, a table 25, a light source control section 26, a light source driving section 27, the light source section 28, and the image processing section 29.

The information reading section 21 is a circuit configured to access the memory 13 of the endoscope 2 connected to the processor 3, and to read information in the memory 13.

The parameter presence determination section 22 is a determination section configured to determine whether or not the control parameter information in the memory 13 can be read by the information reading section 21. Note that since the information reading section 21 cannot access the memory 13 in some cases, the parameter presence determination section 22 determines that the control parameter information in the memory 13 cannot be read when the information reading section 21 cannot access the memory 13.

Accordingly, the parameter presence determination section 22 is a circuit configured to determine whether or not the information in the memory 13 read by the information reading section 21 includes the control parameter information, or whether or not access can be made to an area storing the control parameter information in the memory 13.

The parameter acquisition section 23 is a circuit configured to acquire the control parameter information when the parameter presence determination section 22 determines that the control parameter information is included.

The table reference section 24 is a circuit configured to acquire the control parameter information by referring to the table 25 based on the endoscope ID when the parameter presence determination section 22 determines that the control parameter information is not included.

The table 25 is a retention section configured to store the control parameter information corresponding to the endoscope ID. That is, the table 25 constitutes the retention section configured to retain the control parameter information for controlling operations of the light source section 28 or the image processing section 29.

Even if the information reading section 21 tries to read information in the memory 13 of the endoscope 2, the information reading section 21 cannot access the area storing the control parameter information in the memory 13 of the endoscope 2 in some cases as described above due to a circuit failure of the memory 13 or the like. In such a case, the parameter presence determination section 22 determines that the control parameter information in the memory 13 cannot be read. When the parameter presence determination section 22 determines that the control parameter information in the memory 13 cannot be read, the parameter acquisition section 23 determines that the control parameter information cannot be acquired. When the parameter acquisition section 23 determines that the control parameter information cannot be acquired, the table 25 is not referred to by the table reference section 24.

The light source control section 26 is a circuit configured to receive and retain the control parameter information acquired by the parameter acquisition section 23 or the table reference section 24, and to supply a light source control signal to the light source driving section 27 based on the control parameter information.

The light source control section 26 is configured to determine illuminating light to be used, depending on the observation mode of the endoscope system 1, to generate a light source control signal for controlling a light quantity of the illuminating light, depending on brightness of an endoscope image generated from the image signal from the image pickup section 11, and to output the light source control signal to the light source driving section 27.

The light source driving section 27 is a circuit configured to generate and output a drive signal to the light source section 28, based on the light source control signal from the light source control section 26.

The light source section 28 includes a plurality of light emitting portions 28a to 28d. Each light emitting portion of the light source section 28 is a light emitting diode (LED) that emits light by being driven based on the drive signal from the light source driving section 27.

The light emitting portion 28a is a light emitting device that outputs violet light (V-LED). The light emitting portion 28b is a light emitting device that outputs blue light (B-LED). The light emitting portion 28c is a light emitting device that outputs green light (G-LED). The light emitting portion 28d is a light emitting device that outputs red light (R-LED).

Note that although each of the light emitting portions is a light emitting diode (LED) here, each of the light emitting portions may be a light emitting portion including a laser diode and a fluorescent body that emits light by receiving laser light from the laser diode.

Note further that although the light source section 28 is a light source configured to output four colors of light, violet, blue, green, and red, the light source section 28 may include other one or more light emitting portions that output other one or more colors of light, such as further including a light emitting portion that outputs orange light as a fifth one.

The light outputted by each light emitting portion of the light source section 28 passes through a condenser lens and the like (not shown) and enters an entry face of a proximal end portion of the light guide 12.

When the endoscope system 1 is used in the ordinary light observation mode, the light emitting portion 28b that outputs blue light, the light emitting portion 28c that outputs green light, and the light emitting portion 28d that outputs red light emit light. The ordinary light observation mode is an observation mode in which an endoscope image obtained by irradiating an object with white light is used.

When the endoscope system 1 is used in the narrow-band light observation mode, the light emitting portion 28a that outputs violet light and the light emitting portion 28c that outputs green light emit light. The narrow-band light observation mode here is an observation mode in which an endoscope image obtained by irradiating an object with relatively-short-wavelength light is used.

Note that although the endoscope system 1 of the present embodiment has two observation modes, namely, the ordinary light observation mode and the narrow-band light observation mode, the endoscope system 1 may also have modes such as a narrow-band light observation mode in which narrow-band light other than the above-described narrow-band light is used, a fluorescent light observation mode, and an infrared light observation mode.

The image processing section 29 is a circuit configured to generate an endoscope image by performing predetermined image processing on the image signal from the image pickup section 11 and to output the endoscope image to a monitor (not shown).

The image processing section 29 is configured to receive and retain the control parameter information obtained by the parameter acquisition section 23 or the table reference section 24, as indicated by dotted lines. The image processing section 29 is configured to perform the image processing by using the control parameter information.

Note that other constituent elements of the processor 3, for example, an operation panel, are not shown in FIG. 1, and a description of the elements is omitted.

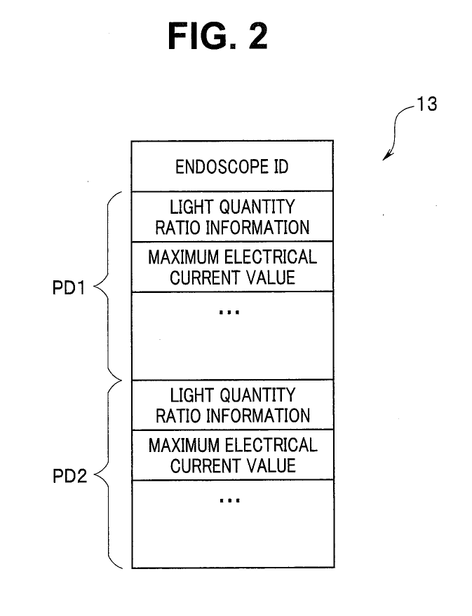

FIG. 2 is a diagram showing an example of the information stored in the memory 13.

The information stored in the memory 13 is at least one of the endoscope ID and the control parameter information. The control parameter information includes a plurality of parameters for controlling the operations of the light source section 28, such as light quantity ratio information and a maximum electrical current value, and a plurality of parameters for controlling the operations of the image processing section 29. The control parameter information is stored in the memory 13 for each observation mode.

The endoscope ID is identification information on the endoscope and information described in a predetermined format.

The light quantity ratio information is information related to control of a light quantity ratio among the plurality of light emitting portions 28a to 28d included in the light source section 28.

Here, an outputted light quantity of each light emitting portion is proportional to an electrical current value flowing in each light emitting portion. Light quantity ratio information (ra:rb:rc:rd) on the four light emitting portions 28a, 28b, 28c, and 28d indicates a ratio among the respective electrical current values flowing in the four light emitting portions 28a, 28b, 28c, and 28d. If each of ra, rb, rc, and rd is expressed by a ratio of each respective electrical current value to a maximum electrical current value MI applied to each light emitting portion, the light quantity ratio information is, for example, (0.0:0.6:1.0:0.4). This case shows that the light emitting portion 28a is not driven, the light emitting portion 28b is driven with an electrical current of 60% of the maximum electrical current value MI, the light emitting portion 28c is driven with an electrical current of the maximum electrical current value MI, and the light emitting portion 28d is driven with an electrical current of 40% of the maximum electrical current value MI.

The maximum electrical current value is a common electrical current value set for the four light emitting portions and indicates a maximum value of an electrical current supplied to each light emitting portion.

The light guide 12 inserted into the endoscope 2 may have a large diameter or a small diameter, depending on a type of the endoscope 2. If the light guide 12 has a small diameter, there is a possibility that the light guide 12 deteriorates due to heat when a large light quantity of illuminating light is cast on an end portion of the light guide 12. Accordingly, information on the maximum electrical current value is included in the control parameter information.

The maximum electrical current value may be set at a lower value than an upper limit value of an electrical current value, at which the light guide 12 does not deteriorate because of heat, whereby the brightness of the endoscope image can be adjusted to be desired brightness.

Note that although the light quantity ratio information and the maximum electrical current value information are shown as the control parameter information for controlling the operations of the light source section 28 here, the control parameter information may also include other information such as a minimum electrical current value indicating a minimum value of an electrical current supplied to each light emitting portion, and duty cycle information used when each light emitting portion is driven by duty control.

As shown in FIG. 2, the memory 13 stores the control parameter information with respect to the two observation modes. The control parameter information used in the ordinary light observation mode is stored in an area PD1 of the memory 13, and the control parameter information in the narrow-band light observation mode is stored in an area PD2 of the memory 13.

FIG. 2 only shows the control parameters for controlling the operations of the light source section 28. However, the control parameters for controlling the operations of the image processing section 29 are also included. The control parameter information for controlling the operations of the image processing section 29 includes information on a coefficient used for a matrix operation to perform the image processing adapted to the light quantity ratio, and the like.

Note that the control parameter information for controlling the operations of both the light source section 28 and the image processing section 29 is used as the control parameter information in the present embodiment. However, the control parameter information may be information for only any one of the light source section 28 and the image processing section 29, or may include control parameter information for controlling operations of another circuit.

Next, the light quantity ratio information, which is one of the control parameter information, will be described.

For generation of an endoscope image, generally, two methods can be used, namely, a simultaneous method and a frame-sequential method.

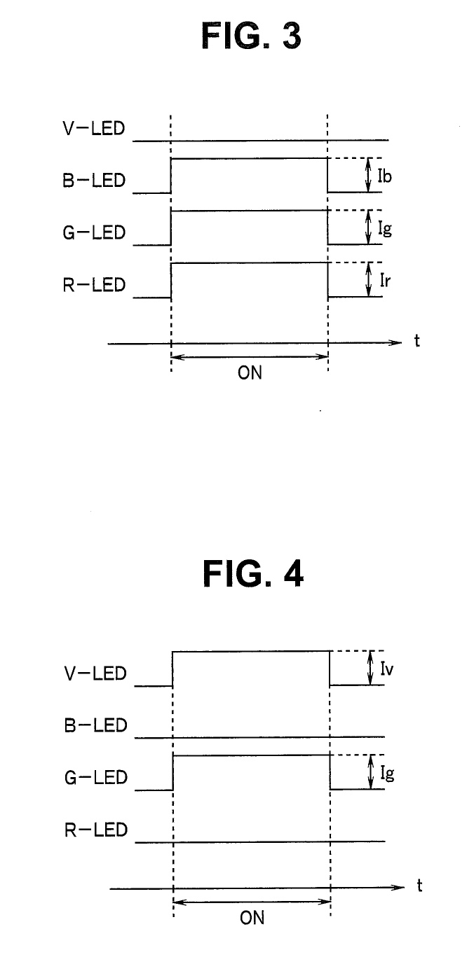

FIG. 3 is a waveform diagram of a drive signal supplied to each light emitting portion in the ordinary light observation mode in case of the simultaneous method. In FIG. 3, a horizontal axis represents time t, and a vertical axis represents electrical current values. In the ordinary light observation mode, a drive signal is supplied to the three light emitting portions 28b, 28c, and 28d, and the three light emitting portions 28b, 28c, and 28d emit light.

In the endoscope 2 for the simultaneous method, a transmitted amount of each color differs from the others due to color filtering properties of the image pickup device. Accordingly, an amount of electrical current supplied to each light emitting portion is adjusted so that light quantities of the three colors are outputted at a predetermined balanced ratio.

When each light emitting portion is turned on/off by duty control of the light source section 28 in the ordinary light observation mode, the light emitting portion 28b of blue color is driven at an electrical current value Ib, the light emitting portion 28c of green color is driven at an electrical current value Ig, and the light emitting portion 28d of red color is driven at an electrical current value Ir during an ON period.

Accordingly, for the light quantity ratio information in FIG. 2, a ratio (V:B:G:R) among the four electrical current values Iv, Ib, Ig, and Ir of the four light emitting portions 28a, 28b, 28c, and 28d is (0:Ib/MI:Ig/MI:Ir/MI).

FIG. 4 is a waveform diagram of a drive signal supplied to each light emitting device in the narrow-band light observation mode in case of the simultaneous method. In FIG. 4, a horizontal axis represents time t, and a vertical axis represents electrical current values. In the narrow-band light observation mode of the present embodiment, a drive signal is supplied to the two light emitting portions 28a and 28c, and the two light emitting portions 28a and 28c emit light.

Accordingly, for the light quantity ratio information in FIG. 2, the ratio (V:B:G:R) among the four electrical current values Iv, Ib, Ig, and Ir of the four light emitting portions 28a, 28b, 28c, and 28d is (Iv/MI:0:Ig/MI:0).

Note that in case of the endoscope system for the frame-sequential method, the endoscope system includes a light source of white light and a rotating filter configured to transmit light outputted from the light source. In case of the frame-sequential method, a received light quantity of each color differs from the others, depending on properties of a plurality of color filters provided to the rotating filter. Accordingly, an amount of electrical current supplied to the light source is adjusted based on a rotated location of the rotating filter so that, for example, light quantities of three colors are outputted at a predetermined balanced ratio.

### (Operation)

Next, operations of the processor 3, which is medical equipment used in combination with the endoscope 2, will be described.

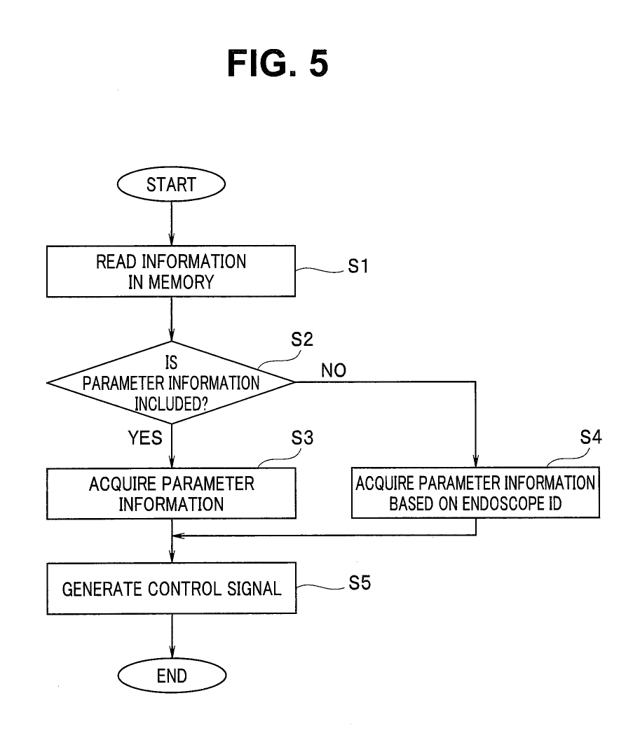

FIG. 5 is a flowchart showing an example of a flow of the operations of the processor 3.

FIG. 5 shows processing performed when the endoscope 2 is connected to the processor 3.

When the endoscope 2 is connected to the processor 3 and a power source of the endoscope 2 is turned on, the processor 3 reads the information stored in the memory 13 (step (hereinafter abbreviated as S) 1). The processing in S1 is performed by the information reading section 21. The memory 13 includes the endoscope ID and the control parameter information, as described above. Accordingly, the information reading section 21 constitutes a reading section configured to read the information stored in the memory 13 from the endoscope 2 including the memory 13 that is capable of storing the control parameter information for controlling the operations of the light source section 28 or the image processing section 29.

The processor 3 determines whether or not the control parameter information can be read from the memory 13, or whether or not the control parameter information is included in the information read from the memory 13 (S2). The processing in S2 is performed by the parameter presence determination section 22.

It is determined that the control parameter information cannot be read from the memory 13 if the processor 3 accesses the memory 13 but cannot access the area storing the control parameter information due to a circuit failure of the memory 13 or the like.

Determination as to whether or not the control parameter information is included in the information read from the memory 13 is performed, for example, as follows.

Since the endoscope ID is data in the predetermined format, it can be determined whether or not the endoscope ID is included, based on whether or not the read information includes data in the predetermined format.

It can also be determined whether or not the control parameter information is included in the information read from the memory 13, based on whether or not information longer than a length of the predetermined format or data in another format than the predetermined format is included.

The determination as to whether or not the control parameter information is included in the information read from the memory 13 may also be made based on presence or absence of predetermined flag information indicating that the control parameter information is included.

Accordingly, the parameter presence determination section 22 constitutes a determination section configured to determine whether or not the control parameter information can be read from the memory. For example, the parameter presence determination section 22 determines whether or not the information read by the information reading section 21 includes the control parameter information for controlling the operations of the light source section 28 or the image processing section 29.

If the information read from the memory 13 includes the control parameter information (S2: YES), the processor 3 acquires the control parameter information and outputs the control parameter information to the light source control section 26 or the image processing section 29 (S3). The processing in S3 is performed by the parameter acquisition section 23.

When the control parameter information cannot be read from the memory 13 or when the information read from the memory 13 does not include the control parameter information (S2: NO), the processor 3 refers to the table 25 based on the endoscope ID, reads the control parameter information from the table 25, and outputs the control parameter information to the light source control section 26 or the image processing section 29 (S4). The processing in S4 is performed by the table reference section 24.

Accordingly, the parameter acquisition section 23 and the table reference section 24 constitute an output section configured to output the control parameter information read by the information reading section 21 when the parameter presence determination section 22 determines that the control parameter information can be read from the memory 13, and to output the control parameter information retained by the table 25 when the parameter presence determination section 22 determines that the control parameter information cannot be read from the memory 13. That is, the parameter acquisition section 23 and the table reference section 24 determine that the control parameter information can be read from the memory 13 if the information read by the information reading section 21 includes the control parameters, and determine that the control parameter information cannot be read from the memory 13 if the information read by the information reading section 21 does not include the control parameters.

For example, when the parameter presence determination section 22 determines that the information read from the memory 13 includes the control parameter information, the parameter acquisition section 23 and the table reference section 24 output the read control parameter information. When the parameter presence determination section 22 determines that the read information does not include the control parameter information, the parameter acquisition section 23 and the table reference section 24 output the control parameter information retained by the table 25.

The light source control section 26 generates a light source control signal based on the control parameter information acquired in S3 or S4, and outputs the light source control signal to the light source driving section 27 (S5). The processing in S5 is performed by the light source control section 26.

For example, if the memory 13 stores the control parameter information, light source control is performed by using the control parameter information read from the memory 13.

The image processing section 29 also performs the image processing based on the control parameter information acquired in S3 or S4.

The light source control or the image processing is also performed by using the control parameter information read from the memory 13 when the memory 13 stores the control parameter information and the endoscope ID. That is, if the parameter presence determination section 22 determines that the information read from the memory 13 includes the control parameter information and if the information read from the memory 13 includes information on the type of the endoscope 2, the parameter acquisition section 23 outputs the control parameter information, but the table reference section 24 does not output the control parameter information.

Accordingly, the control parameter information stored in the memory 13 is given priority, and control of the operations of the light source section 28 or the image processing section 29 is performed by using the control parameter information read from the memory 13. The endoscope ID is not used for control of the operations of the light source section 28 or the image processing section 29.

If the memory 13 does not store the control parameter information and if the information read from the memory 13 includes only the endoscope ID, the light source control is performed by using the endoscope ID read from the memory 13. That is, the control parameter information is acquired by referring to the table 25 based on the endoscope ID read from the memory 13, and the light source control or the image processing is performed by using the acquired control parameter information.

Note that although FIG. 5 shows a flow of the operations of each circuit included in the processor 3, part or all of functions of the plurality of circuits included in the processor 3 may be implemented by using a software program. In this case, the processor 3 includes a central processing unit (CPU), and the central processing unit (CPU) executes each step in the processing shown in FIG. 5.

### (Effects)

As described above, according to the above-described embodiment, it is possible to provide medical equipment that can be used in combination with any of an endoscope having endoscope identification information and an endoscope having control parameter information, and a medical equipment system

Particularly in some cases, an operator uses the endoscope 2 having the endoscope ID and desires to cause the light source control section 26 or the image processing section 29 to operate by using other control parameter information than the control parameter information that can be acquired by referring to the table 25 based on the endoscope ID. In such a case, if the other control parameter information is added to the memory 13, the user can cause the light source control section 26 or the image processing section 29 to operate by using the other control parameter information than the control parameter information that can be acquired by referring to the table 25 based on the endoscope ID.

Conventionally, for example, even if a newly developed endoscope has an endoscope ID, the newly developed endoscope cannot be used in a system in which endoscope ID information is transmitted and received if the endoscope ID of the new endoscope and light source control parameter information on the new endoscope are not registered with a processor to be combined.

However, according to the above-described embodiment, if control parameter information is registered in a memory of the newly developed endoscope, the processor preferentially uses the control parameter information in light source control or image processing, and accordingly, the newly developed endoscope can be used in combination with the processor.

Alternatively, when an endoscope maker desires to use the conventional endoscope 2 having the endoscope ID to perform other light source control or image processing, the endoscope maker can easily cause the processor 3 to perform the other light source control or image processing by using the endoscope 2 having the endoscope ID, by adding control parameter information to the memory 13.

Moreover, even in a case where, for example, control parameter information cannot be read due to a circuit failure of a memory or the like even if the control parameter information is registered in the memory of an endoscope, the control parameter information can be acquired by referring to the table 25 based on an endoscope ID.

The present invention is not limited to the above-described embodiment. Various changes, modifications, and the like can be made without changing the scope of the present invention.

For example, the light quantity ratio information does not need to be a ratio among electrical current values driving the respective light emitting portions, but may be a ratio among light quantities of light actually emitted by the respective light emitting portions.

In this case, light quantity sensors 30a, 30b, 30c, and 30d configured to receive light from the respective light emitting portions are provided in vicinity of the four light emitting portions 28a, 28b, 28c, and 28d, respectively. The light source control section 26 drives the four light emitting portions 28a, 28b, 28c, and 28d so that the light quantities of the respective light emitting portions sensed by the light quantity sensors 30a, 30b, 30c, and 30d have a light quantity ratio indicated by the light quantity ratio information.

What is claimed is:

1. Medical equipment used in combination with an endoscope, comprising:
a table configured to retain first control parameter information for controlling operations of a light source section or an image processing section;
a reading circuit configured to read information stored in a memory from the endoscope including the memory that is capable of storing second control parameter information for controlling the operations of the light source section or the image processing section;
a determination circuit configured to determine whether or not the second control parameter information can be read from the memory; and
an output circuit configured to output the second control parameter information read by the reading circuit when the determination circuit determines that the second control parameter information can be read from the memory, and to output the first control parameter information retained by the table when the determination circuit determines that the second control parameter information cannot be read from the memory.

2. The medical equipment according to claim 1, wherein the determination circuit determines that the second control parameter information can be read from the memory if the information read by the reading circuit includes the second control parameter, and determines that the second control parameter information cannot be read from the memory if the information read by the reading circuit does not include the second control parameter.

3. The medical equipment according to claim 1, wherein the table stores the first control parameter information corresponding to information on a type of the endoscope,
the information stored in the memory includes the information on the type of the endoscope, and
when the determination circuit determines that the second control parameter information cannot be read from the memory, the output circuit refers to the table based on the information on the type of the endoscope and outputs the first control parameter information read from the table.

4. The medical equipment according to claim 3, wherein when the determination circuit determines that the second control parameter information can be read from the memory and when the information includes the information on the type of the endoscope, the output circuit outputs the second control parameter information.

5. The medical equipment according to claim 1, wherein the first and second control parameter information for controlling the operations of the light source section is information related to control of a light quantity ratio among a plurality of light sources included in the light source section.

6. A medical equipment system, comprising:
an endoscope; and
medical equipment used in combination with the endoscope,
wherein the medical equipment includes:
a table configured to retain first control parameter information for controlling operations of a light source section or an image processing section;
a reading circuit configured to read information stored in a memory from the endoscope including the memory that is capable of storing second control parameter information for controlling the operations of the light source section or the image processing section;
a determination circuit configured to determine whether or not the second control parameter information can be read from the memory; and
an output circuit configured to output the second control parameter information read by the reading circuit when the determination circuit determines that the second control parameter information can be read from the memory, and to output the first control parameter information retained by the table when the determination circuit determines that the second control parameter information cannot be read from the memory.

7. Medical equipment used in combination with an endoscope, comprising:
a table configured to retain first control parameter information for controlling operations of a light source section or an image processing section; and
a processor including hardware,
wherein the processor is configured to:
read information stored in a memory from the endoscope including the memory that is capable of storing second control parameter information for controlling the operations of the light source section or the image processing section;
determine whether or not the second control parameter information can be read from the memory; and
output the read second control parameter information when it is determined that the second control parameter information can be read from the memory, and output the first control parameter information retained by the table when it is determined that the second control parameter information cannot be read from the memory.
